(19) United States
(12) Patent Application Publication
(10) Pub. No.: US 2003/0060805 A1
(43) Pub. Date: Mar. 27, 2003

(54) Handheld rotational device for use during surgeries

(75) Inventors: Alfonso F. del Granado, Chicago, IL (US); Francisco M. del Granado, Darien, IL (US); Zachary J. Mottl, Darien, IL (US)

Correspondence Address: BANNER & WITCOFF, LTD., TEN SOUTH WACKER DRIVE, SUITE 3000, CHICAGO, IL, 60606, US

(73) Assignee: ZF International, L.L.C., Lyons, IL

(21) Appl. No.: 10/255,212

(22) Filed: Sep. 26, 2002

Related U.S. Application Data
(60) Provisional application No. 60/325,090, filed on Sep. 26, 2001.

(51) Int. Cl. A61M 1/00

(52) U.S. Cl. 604/540; 606/180; 604/35

(57) ABSTRACT

Disclosed is a motorized apparatus for rotating suction tubes during a medical surgical operation. The apparatus consists of a lightweight motor, motor housing and drive assembly capable of automatically rotating a vacuum curette or similar suction tube at the bodily sight of a surgical procedure. In practice, the motor generates rotational force capable of rotating the suction tube, avoiding the risks of repetitive stress injuries' to surgeons and surgical assistants.

This application claims the benefit of U.S. Provisional Application Serial No. 60/325,090, filed Sep. 26, 2001.

### FIELD OF THE INVENTION

This invention relates to surgical devices. Specifically, the invention relates to a motorized apparatus for rotating suction tubes used in the evacuation of solutions and tissues from a surgical operation. The device is particularly suited for rotating vacuum curettes within the human body during endoscopic surgery. In this application, the invention may be used to safely and efficiently evacuate blood or other fluids and tissues from the site of a surgical procedure.

### BACKGROUND OF THE INVENTION

Surgery can cause fluids and tissues to be released or introduced at the bodily site of an operation. When these solutions are unwanted or in excessive quantities, a surgeon will periodically evacuate the site of an operation during a surgical procedure. In practice, medical personnel employ vacuum pumps and tubing to suction solutions out of the body. Generally, flexible suction tubing connects the vacuum pump to a handle that is held by the surgeon or surgical assistant whenever suctioning is required. The handle, in turn, is connected to a rigid or semi-rigid vacuum curette. The curette is attached so that it can be rotated relative to the handle, and is adapted to be inserted into the body to provide an inlet for suctioned solutions. When suctioned, these solutions pass through the inlet tip of the curette and are carried through the tubing to a reservoir associated with the pump.

Typically, the curette must be rotated while it is inserted in the body to efficiently evacuate solutions from the site of an operation. Until now, the curette has been rotated manually. Specifically, when suction was required and the vacuum curette was inserted in the body, the surgeon or surgical assistant rotated the curette constantly by spinning the curette between the fingers of the hand holding the tube-handle-curette assembly. Depending on the surgical procedure and the patient's condition, suction and manual rotation of the vacuum curette could be required numerous times during a surgery and over extended durations.

In this way, previously known suctioning devices required medical personnel to rotate the curette for sustained and repetitive periods of time. These physical demands placed surgeons and surgical staff at risk for developing injuries to the hand and wrist, like carpal tunnel syndrome. Such injuries can be painful and potentially debilitating to a surgeon's medical practice. Thus, what is needed is a reliable, convenient and cost-effective way to automate the rotation of the curette within the vicinity of a surgical procedure.

### BRIEF DESCRIPTION OF THE DRAWINGS

FIG. 1 is a schematic view of a surgical system including the handheld rotational device in accordance with a preferred embodiment of the present invention in relation to a patient's body.

FIG. 2 is a side view of the handheld rotational device of the present invention.

FIG. 3 is a cross-section view the handheld rotational device of FIG. 2.

FIG. 4 is a partial end view of the drive assembly of the handheld rotational device of the present invention.

### DESCRIPTION OF THE INVENTION

The device of the present invention automates the rotation of a vacuum curette during surgical procedures. The device is adapted to comfortably fit in a user's hand and provide controlled rotation of the vacuum curette. More particularly, the invention uses a motor and drive system to transmit rotational force to the curette. In this way, the curette can be rotated while providing suction at the bodily site of a surgical procedure, without the risk of injuries to the user's hand and wrist.

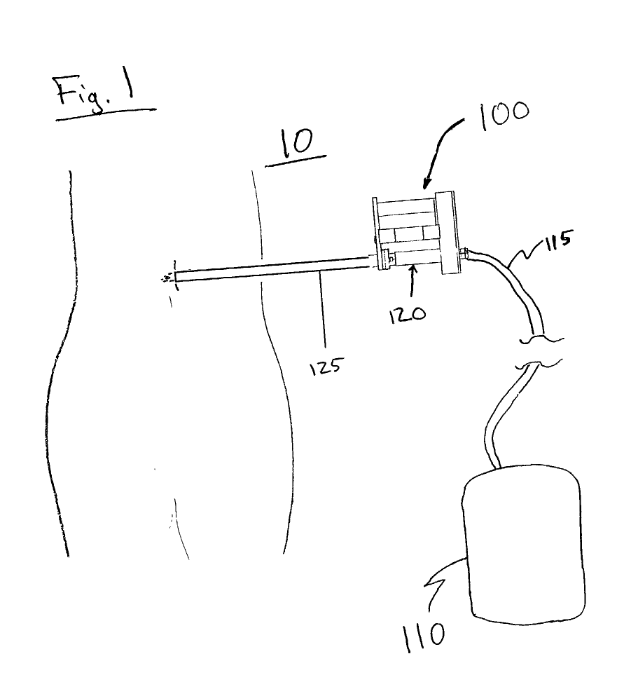

As shown in FIG. 1, a system 10 to suction solutions and tissues out of the body is provided. The system 10 generally comprises a device 100 linked to a medical vacuum pump 110 by flexible tubing 115. Power for device 100 can be transmitted by connections associated with flexible tubing 115 or by separate connections to appropriate power supplies that are generally understood in the art. Flexible tubing 115 is attached to device 100 at a fixed, barbed connector (not visible), which in turn is connected to rotational tube 120 running through the device 100. A vacuum curette 125 generally known in the art is connected to the rotational tube 120 opposite the device's connection with flexible tubing 115. Within device 100, rotational force is transmitted to rotational tube 120. In practice, the device allows flexible tubing 115 to remain fixed relative to the device, while curette 125 is automatically rotated by rotational force applied to rotational tube 120 from within device 100. As shown in FIG. 1, the vacuum curette 125 is inserted into a patient's body to evacuate solutions and tissues during a surgical procedure. The vacuum created by vacuum pump 110 suctions solutions from within the body through rotating curette 125, rotational tube 120 and flexible tubing 115. As is commonly understood in the art, suctioned fluids and tissues are ultimately collected in a reservoir associated with pump 110 after passing through flexible tubing 115.

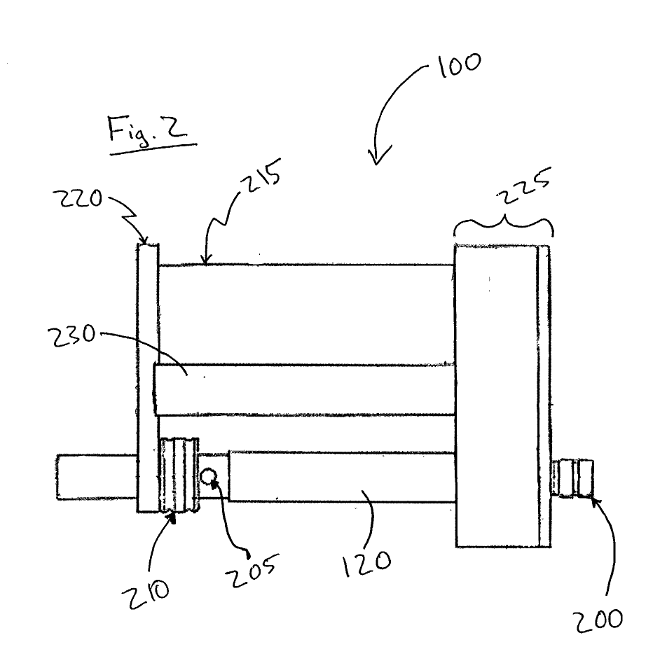

A side profile of the device 100 is shown in FIG. 2. Rotational tube 120 can be seen running through device 100. Rotational tube 120 is sealeably connected to fixed, barbed connector 200 within device 100. Referring now to FIGS. 1 and 2 collectively, one will understand that a user can comfortably hold device 100 by grasping motor housing 215. In this orientation, the device of the present invention is held in one hand, flexible tubing 115 is affixed to device 100 at connector 200, and rotational tube 120 with vacuum curette 125 can be automatically rotated at the site of an operation.

Continuing to refer to FIGS. 1 and 2 as a group, vent hole 205 and slideable collar 210 can be seen on rotational tube 120. A user can slide collar 210 on rotational tube 120 from a position fully exposing vent hole 205, to a position fully covering vent hole 205. In this way, a user can control the vacuum suction at the inlet of curette 125 when a vacuum is applied to the device through flexible tubing 115. In particular, when vent hole 205 is fully covered by slideable collar 210, a uniform vacuum is transmitted from pump 110 to the tip of curette 125, as if flexible tubing 115, rotational tube 120 and curette 125 were a single length of solid tubing. However, when collar 210 is slideably moved to expose vent hole 205, the suction force generated by pump 110 is vented before reaching the curette 125. Vent hole 205 and slideable collar 210 thus allow a user to quickly turn on or off the vacuum suction at the inlet tip of curette 125. In an alternative embodiment, an electrical switch on the device 100 or the pump 110 may be utilized to turn the vacuum suction on or off. In yet another alternative, a plurality of vent holes may be provided to adjust the suction pressure generated by the system 10.

As further seen in FIG. 2, in an exemplary embodiment of the present invention rotational tube 120 is secured in relation to sturdy and lightweight motor housing 215. At either end of motor housing 215, end plates 220 and 225 are affixed to securely hold the components of device 100 in relation to one another. Rotational tube 120 passes through the device by way of one or more bearings or bushings known to those in the art that reduce friction and wear when rotational tube 120 is rotating. Reinforcement member 230 is optionally included to provide structural reinforcement to device 100, as well as a convenient location for various power controls and a channel for power connections for the device that will be apparent to those in the art.

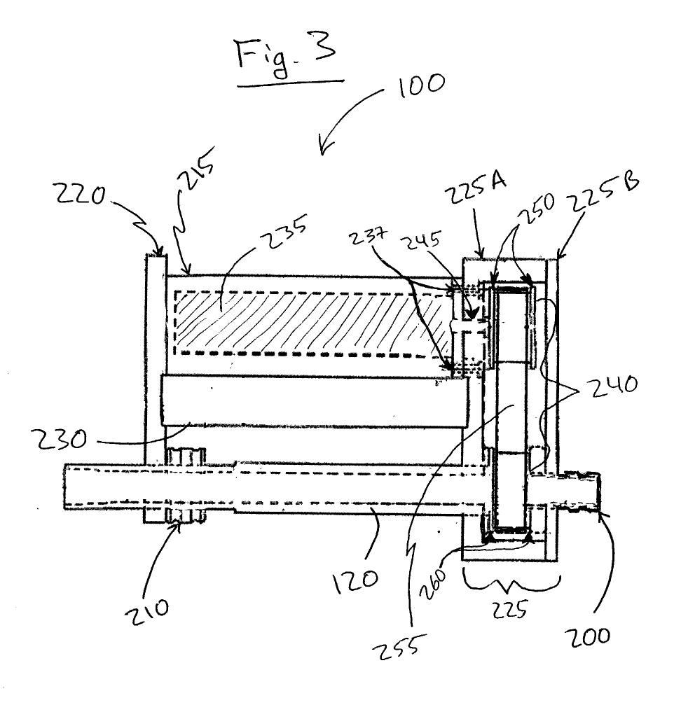

A cross-section of device 100 is illustrated in FIG. 3. End cap 225 is shown composed of two parts 225A and 225B. In this configuration, end cap 225 provides a point of attachment for a motor 235, as well as an accessible, but enclosed, compartment for a drive assembly 240. Motor 235 is affixed to end plate 225A by one or more fasteners 237. Drive assembly 240 includes motor shaft 245, drive pulley 250, drive belt 255 and transfer pulley 260. In practice, drive pulley 250 is affixed to the shaft 245 of motor 235. When activated, motor 235 produces a rotational force that is transmitted to drive pulley 250 through motor shaft 245.

Correspondingly, transfer pulley 260 is securely affixed to rotational tube 120. In turn, rotational force from drive pulley 250 is transferred to transfer pulley 260 and rotational tube 120 by way of drive belt 255. Thus, motor 235 is capable of rotating rotational tube 120 through the drive assembly 240. One skilled in the art will recognize that drive assembly 240 can be composed of a system of various and/or numerous belts and pulleys, or even a variety of toothed gears. In this way, drive assembly 240 can be adapted to transfer rotational force at a variety of drive ratios, depending on the number and size of pulleys, belts or gears employed. This flexibility allows the device to accommodate a wide range of motor speeds and surgical demands. Thus, for example, a motor operating at 28 revolutions per minute (RPM) could rotate rotational tube 120 and vacuum curette 125 at a rate of 23 RPM through a reducing gear ratio in drive assembly 240.

Those skilled in the art will further understand that motor 235 can be any one of a number of various units capable of imparting rotational motion. For example, motor 235 can be a small electric motor powered by batteries or standard alternating current. Alternatively, motor 235 can be a compact, air, liquid or other fluid driven turbine, conveniently powered by a pressurized or circulating fluid source. In yet another alternative, motor 235 can consist of a narrow, flexible drive shaft connected to a freestanding electric motor or air driven turbine unit. In this alternative, the comparatively bulky or heavy parts of the motor 235 can be distributed relative device 100.

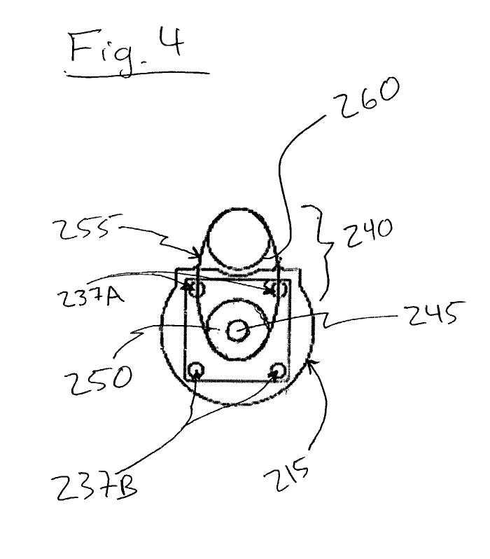

FIG. 4 shows a partial end-view of drive assembly 240. As described, drive pulley 250 is attached to motor shaft 245, and is capable of transferring rotational force to transfer pulley 260 by way of belt 255. Fasteners 237A and 237B are provided to hold motor 235 (not visible) in place relative to motor housing 215.

As seen in FIGS. 1-4, it is now possible, with the invented surgical device 100, to automate the rotation of a vacuum curette and avoid the painful and debilitating injuries associated with previously known suction devices. In practice, users can easily and comfortably hold the device 100 by grasping motor housing 215 in one hand. Vacuum suction at the tip of vacuum curette 125 can be controlled by sliding collar 210 in relation to vent hole 205. Power and controls for motorization of the device can conveniently be located within reinforcement member 230. The device can readily be operated with one hand to automatically rotate a vacuum curette and evacuate the bodily site of a surgical procedure.

A distinct advantage of this device is its efficient automation of rotating suction tubes during surgery. The invention is suitable for any medical procedure that requires vacuum suctioning, including at least, endoscopic surgical procedures. Specifically, the device is well-suited for, among others, termination of pregnancy procedures.

What is claimed is:

1. A rotational medical device for rotating a suction tube during a medical surgery, comprising in combination:
(a) a housing;
(b) a motor affixed to the housing capable of producing a rotational force;
(c) a rotatable suction tube positioned in the housing; and
(d) a drive assembly adapted to transmit rotational force produced by the motor to the rotatable suction tube.

2. The rotational medical device of claim 1 further comprising:
(e) a connector for receiving flexible tubing fixed to one end of the device and adapted to sealeably connect to the rotatable suction tube.

3. The rotational medical device of claim 2 further comprising:
(f) a vacuum curette fixed to the rotatable suction tube opposite the connector.

4. The rotational medical device of claim 3 further comprising:
(g) flexible tubing attached to the device by the connector.

5. The rotational medical device of claim 4 further comprising:
(h) a vacuum pump connected to the device by the flexible tubing.

6. The rotational medical device of claim 5 further comprising:
(i) a slideable collar and one or more vent holes on the rotatable suction tube adapted to control the vacuum pressure within the vacuum curette.

7. The rotational medical device of claim 1 wherein the motor is an electric motor.

8. The rotational medical device of claim 1 wherein the motor is a fluid driven turbine.

9. The rotational medical device of claim 1 wherein the drive assembly includes at least a pair of pulleys.

10. The rotational medical device of claim 1 wherein the drive assembly includes at least a pair of gears.

11. The rotational medical device of claim 1 wherein the drive assembly includes one or more belts.

12. The rotational medical device of claim 1 wherein the drive assembly includes one or more chains.

13. A method for automatically rotating a suction tube at the bodily site of a medical surgery comprising the steps of:
(a) positioning a suction tube within a patient's body, the suction tube being connected to a rotatable tube capable of being rotated with a motor and drive assembly; and
(b) activating the motor so that rotational force is transmitted through the drive assembly to the suction tube inserted within the patient's body.

14. The method of claim 13, wherein the step of positioning the suction tube is suited for termination of pregnancy procedures.

15. A rotational medical device for rotating a suction tube during medical surgery, comprising in combination:
(a) a housing;
(b) a rotatable suction tube positioned in the housing;
(c) A means for producing a rotational force; and
(d) A means for transferring the rotational force to the rotatable suction tube.

16. The rotational medical device of claim 15 further comprising:
(e) a connector for receiving flexible tubing fixed to one end of the device and adapted to sealeably connect to the rotatable suction tube.

17. The rotational medical device of claim 16 further comprising:
(f) a vacuum curette fixed to the rotatable suction tube opposite the connector.

18. The rotational medical device of claim 17 further comprising:
(g) flexible tubing attached to the device by the connector.

19. The rotational medical device of claim 18 further comprising:
(h) a vacuum pump connected to the device by the flexible tubing.

20. The rotational medical device of claim 19 further comprising:
(i) a slideable collar and one or more vent holes on the rotatable suction tube adapted to control the vacuum pressure within the vacuum curette.
